(19) United States
(12) Patent Application Publication
(10) Pub. No.: US 2019/0022104 A1
(43) Pub. Date: Jan. 24, 2019

(54) APPLICATION OF PIRENZEPINE FOR TREATING SEPSIS

(71) Applicant: Zhen Wang, Beijing (CN)

(72) Inventor: Zhen Wang, Beijing (CN)

(21) Appl. No.: 16/070,065

(22) PCT Filed: Jan. 13, 2017
(86) PCT No.: PCT/CN2017/071118

§ 371 Date: Jul. 13, 2018

(30) Foreign Application Priority Data
Jan. 14, 2016 (CN) 201610025409.8

(51) Int. Cl. A61K 31/5513; A61K 9/00; A61P 29/00

(52) U.S. Cl. CPC A61K 31/5513; A61P 29/00; A61K 9/0019

(57) ABSTRACT

Provided is a method of treating sepsis, which includes administering pirenzepine or a pharmaceutically acceptable salt thereof.

### CROSS REFERENCE TO RELATED APPLICATIONS

The present application is a national application of PCT/CN2017/071118 filed on Jan. 13, 2017, which claims the priority of the Chinese Patent Application No. 201610025409.8 filed on Jan. 14, 2016. The Chinese Patent Application No. 201610025409.8 is incorporated herein by reference as part of the disclosure of the present application.

### FIELD OF THE INVENTION

The present disclosure relates to the field of medical technology, in particular the use of pirenzepine in the treatment of sepsis.

### BACKGROUND OF THE INVENTION

Pirenzepine is a selective anticholinergic drug with high affinity for muscarinic receptors of gastric parietal cells and low affinity for muscarinic receptors of smooth muscle, myocardium, salivary gland and the like. Therefore, when used in a common therapeutic dose, it only inhibits gastric acid secretion, and rarely has side effects of other anticholinergic drugs on the pupil, gastrointestinal smooth muscle, heart, salivary gland, bladder muscle, and the like. Increased doses can suppress salivary secretion, and only large doses can inhibit gastrointestinal smooth muscle and cause tachycardia.

Pirenzepine cannot penetrate the blood-brain barrier and thus does not affect the central nervous system. After oral administration, intramuscular injection, or intravenous injection of pirenzepine, both basic gastric acid secretion and gastric acid secretion caused by exogenous pentagastrin and insulin were inhibited. Pirenzepine has little effect on the pH of gastric juices. It mainly reduces the secretion of gastric juices (including pepsinogen and pepsin), thus reducing the maximum acid secretion and the highest acid secretion in the stomach. Its oral absorption is not complete, tmax is 2-3 hours, absolute bioavailability is about 26% (±4,6%), and food has an impact on absorption. Except for brain and embryonic tissues, pirenzepine was distributed in other organs and skeletal muscle, with liver and kidney have the highest concentrations, followed by spleen and lung, and heart, skin, muscle, and blood have lower concentrations. The plasma protein binding rate is about 10%. It is rarely metabolized in the body, and is mostly excreted through the kidney and biliary tract as the original compound. Plasma t½ is 10-12 hours. Within 24 hours, it is excreted with feces as the original compound. Although administered for 3-4 days, pirenzepine can be excreted completely all the time and no accumulation is observed.

Pirenzepine is mainly used to treat gastric and duodenal ulcers, can significantly relieve pain in patients, and reduce the use of antacids. The healing rate of the ulcer is approximately 70%-94%. The therapeutic effect is similar to that of cimetidine and is superior to sodium carbenoxolone. The combination of pirenzepine with cimetidine can enhance the effect of inhibiting the secretion of gastric acid.

Pirenzepine acts as an acid suppressant. Clinically, it is mainly used for various acid-related diseases such as duodenal ulcer, gastric ulcer, gastro-esophageal reflux disease, hyperacid gastritis, stress ulcer, acute gastric mucosal bleeding, gastrinoma and so on.

Sepsis, including severe sepsis and septic shock, is a systemic inflammatory response to infection or trauma. There is no relevant research in the prior art for the use of pirenzepine in the treatment of sepsis diseases.

### SUMMARY OF THE INVENTION

A brief summary of the invention is given below in order to provide a basic understanding of some aspects of the invention. It should be understood that this summary is not an exhaustive summary of the invention. It is not intended to identify the key or important parts of the invention, nor is it intended to limit the scope of the invention. Its purpose is merely to present some concepts in a simplified form as a prelude to a more detailed description that will be discussed later.

The present disclosure is to provide a new use of pirenzepine or a pharmaceutically acceptable salt thereof in the treatment of sepsis (including sepsis shock) in order to address the above-mentioned deficiencies of the prior art. Preferably, the pirenzepine is administered by intraperitoneal injection, intramuscular injection, intravenous injection or oral administration. More preferably, said pirenzepine is administered by intraperitoneal injection, wherein the amount per injection is 0.1-20 mg/kg, more preferably 3 mg/kg, based on pirenzepine, dissolved in 0.05 ml physiological saline. More preferably, the pirenzepine is administered by intramuscular or intravenous injection or oral administration in an amount of 10-1000 mg/day, preferably 18-150 mg/day, based on pirenzepine, administered once or several times a day, preferably 1-3 times a day.

Compared with the prior art, the beneficial effects of the invention are:

Through experimental analysis, the present invention finds out that pirenzepine has a certain effect in the treatment of sepsis (including sepsis shock) diseases and can be widely used in the medicament for the treatment of sepsis (including sepsis shock) clinically.

### DETAILED DESCRIPTION OF THE INVENTION

In a first embodiment, disclosed herein is the use of pirenzepine or a pharmaceutically acceptable salt thereof in the manufacture of a medicament for the treatment of sepsis, including sepsis shock. More specifically, disclosed herein is the use of pirenzepine or a pharmaceutically acceptable salt thereof in the manufacture of a medicament for treating sepsis in a subject, wherein said medicament comprises a therapeutically effective amount of pirenzepine or a pharmaceutically acceptable salt thereof. More specifically, the subject is human.

Preferably, the medicament is administered by intraperitoneal injection, intramuscular injection, intravenous injection or oral administration.

Preferably, the medicament is administered by intraperitoneal injection, wherein the amount per injection is 0.1-20 mg/kg, preferably 0.3 mg/kg, 0.6 mg/kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, 20 mg/kg, based on pirenzepine.

Preferably, the medicament is administered by intramuscular or intravenous injection in an amount of 10-1000 mg/day, preferably 18-150 mg/day, once daily or in divided doses, based on pirenzepine.

Preferably, the medicament is administered orally, in an amount of 10-1000 mg/day, preferably 18-150 mg/day, one to three times daily, based on pirenzepine.

Preferably, the medicament is in a unit dosage form of a solid or liquid formulation such as a capsule, a pill, a tablet, a lozenge, a troche, a collosol, a powder, a solution, a suspension, or an emulsion.

Preferably, the amount of pirenzepine or a pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 mg, based on pirenzepine.

In a second embodiment, disclosed herein is a method of treating sepsis, including septic shock, comprising administering pirenzepine or a pharmaceutically acceptable salt thereof. More specifically, disclosed herein are a method of treating sepsis in a subject, comprising administering to the subject a therapeutically effective amount of pirenzepine or a pharmaceutically acceptable salt thereof. More specifically, the subject is human.

Preferably, said pirenzepine or a pharmaceutically acceptable salt thereof is administered by intraperitoneal injection, intramuscular injection, intravenous injection or oral administration.

Preferably, the pirenzepine or a pharmaceutically acceptable salt thereof is administered by intraperitoneal injection, wherein the amount per injection is 0.1-20 mg/kg, preferably 0.3 mg/kg, 0.6 mg/kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, 20 mg/kg, based on pirenzepine.

Preferably, the pirenzepine or a pharmaceutically acceptable salt thereof is administered by intramuscular or intravenous injection in an amount of 10-1000 mg/day, preferably 18-150 mg/day, once or several times a day, based on pirenzepine.

Preferably, said pirenzepine or a pharmaceutically acceptable salt thereof is orally administered in an amount of 10-1000 mg/day, preferably 18-150 mg/day, 1-3 times per day, based on pirenoxime.

Preferably, said pirenzepine or a pharmaceutically acceptable salt thereof is in a unit dosage form of a solid or liquid formulation, such as a capsule, a pill, a tablet, a lozenge, a troche, a collosol, a powder, a solution, a suspension or an emulsion.

Preferably, the amount of pirenzepine or a pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 mg, based on pirenzepine.

In a third embodiment, disclosed herein is pirenzepine or a pharmaceutically acceptable salt thereof for use in the treatment of sepsis, including sepsis shock. More specifically, disclosed herein is pirenzepine or a pharmaceutically acceptable salt thereof for use in the treatment of sepsis in a subject comprising administering to the subject a therapeutically effective amount of pirenzepine or a pharmaceutically acceptable salt thereof. More specifically, the subject is human.

Preferably, said pirenzepine or a pharmaceutically acceptable salt thereof is administered by intraperitoneal injection, intramuscular injection, intravenous injection or oral administration.

Preferably, pirenzepine or a pharmaceutically acceptable salt thereof is administered by intraperitoneal injection, wherein the amount per injection is 0.1-20 mg/kg, preferably 0.3 mg/kg, 0.6 mg/kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, 20 mg/kg, based on pirenzepine.

Preferably, pirenzepine or a pharmaceutically acceptable salt thereof is administered by intramuscular or intravenous injection in an amount of 10-1000 mg/day, preferably 18-150 mg/day, once or several times a day, based on pirenzepine.

Preferably, the pirenzepine or a pharmaceutically acceptable salt thereof is orally administered in an amount of 10-1000 mg/day, preferably 18-150 mg/day, 1-3 times per day, based on pirenzepine.

Preferably, the pirenzepine or a pharmaceutically acceptable salt thereof is in a unit dosage form of a solid or liquid formulation, such as a capsule, a pill, a tablet, a lozenge, a troche, a collosol, a powder, a solution, a suspension or an emulsion.

Preferably, the amount of pirenzepine or a pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 mg, based on pirenzepine.

In a fourth embodiment, disclosed herein is the use of pirenzepine or a pharmaceutically acceptable salt thereof for the treatment of sepsis, including sepsis shock. More specifically, disclosed herein is the use of pirenzepine or a pharmaceutically acceptable salt thereof for treating sepsis in a subject comprising administering to said subject a therapeutically effective amount of pirenzepine or a pharmaceutically acceptable salt thereof. More specifically, the subject is human.

Preferably, the pirenzepine or a pharmaceutically acceptable salt thereof is administered by intraperitoneal injection, intramuscular injection, intravenous injection or oral administration.

Preferably, pirenzepine or a pharmaceutically acceptable salt thereof is administered by intraperitoneal injection, wherein the amount per injection is 0.1-20 mg/kg, preferably 0.3 mg/kg, 0.6 mg/kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, 20 mg/kg, based on pirenzepine.

Preferably, the pirenzepine or a pharmaceutically acceptable salt thereof is administered by intramuscular or intravenous injection in an amount of 10-1000 mg/day, preferably 18-150 mg/day, once or several times a day, based on pirenzepine.

Preferably, the pirenzepine or a pharmaceutically acceptable salt thereof is orally administered in an amount of 10-1000 mg/day, preferably 18-150 mg/day, 1-3 times per day, based on pirenoxime.

Preferably, the pirenzepine or a pharmaceutically acceptable salt thereof is in a unit dosage form of a solid or liquid formulation, such as a capsule, a pill, a tablet, a lozenge, a troche, a collosol, a powder, a solution, a suspension or an emulsion.

Preferably, the amount of pirenzepine or a pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 mg, based on pirenzepine.

### Definition

The term “treating/treatment” refers to a method for obtaining a beneficial or desired clinical result. For the purposes of the present disclosure, the beneficial or desired clinical results include, but are not limited to, symptomatic relief, reduction in disease severity, stable (e.g., no worsened) disease state, delayed or slowed disease progression, improved or alleviated disease state, and remission (partial or overall), whether detectable or undetectable. “Treating/treatment” may also refer to prolonged survival compared to the expected survival if not receiving treatment or therapy. Thus, “treating/treatment” is an intervention that is intended to alter the pathology of a condition. In particular, treatment can directly prevent, slow down, or reduce the pathology of cell degeneration or injury, such as the pathology of organ cells in sepsis, or can make cells more sensitive to treatment or therapy using other therapeutic agents.

The terms “therapeutically effective amount”, “effective amount”, or “sufficient amount” refer to an amount of an active agent sufficient to cause a particular biological state, effect, and/or response, specifically, in the context of the present disclosure, refer to an amount sufficient to achieve a desired result when administered to a subject, including a mammal, such as a human, for example an amount effective to treat sepsis. The effective amount of an agent described herein may vary depending on factors such as the subject's disease state, age, sex, and body weight. As will be understood by the skilled artisan, dosages or treatment regimens may be adjusted to provide optimal therapeutic response. For example, in the present disclosure, pirenzepine or a pharmaceutically acceptable salt thereof is administered in an amount of 0.1-20 mg/kg, preferably 0.3 mg/kg, 0.6 mg/kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, 20 mg/kg, based on pirenzepine. Alternatively, the dosage is 10-1000 mg/day, preferably 18-150 mg/day, once or several times a day, based on pirenzepine.

In addition, a therapeutically effective amount of a subject's treatment regimen may consist of a single administration or include a series of administrations. The length of the treatment period depends on various factors such as the severity of the disease, the age of the subject, the concentration of the agent, the patient's response to the agent, or a combination thereof. It will also be understood that the effective dosage of the agent for treatment may be increased or decreased during the course of a particular treatment regimen. Dose changes can be produced and become apparent by standard diagnostic assays known in the art. In one aspect, the agents of the present disclosure can be administered before, during, or after treatment with conventional therapies for the disease or condition in question, such as sepsis.

The term “subject” refers to any member of the animal kingdom, typically a mammal. The term “mammal” refers to any animal classified as a mammal, including humans, other higher primates, livestock and farm animals, and a zoo, sport or pet animal such as dogs, cats, cows, horses, sheep, pigs, goats, rabbits and the like. In general, mammals are humans.

The term “pharmaceutically acceptable salts” refers to salts that are suitable for use in contact with human or animal tissues without undue toxicity, irritation, allergic response, etc. within the scope of sound medical judgment, and provides reasonable benefit/risk ratio. “Pharmaceutically acceptable salt” means any at least substantially non-toxic salt or ester salt of a compound disclosed herein that, upon administration to a recipient, is capable of providing, either directly or indirectly, a compound disclosed herein or a metabolite or residue thereof with inhibitory activity.

Pharmaceutically acceptable salts are well known in the art. For example, pharmaceutically acceptable salts are described by S. M. Berge et al. in detail in J. Pharmaceutical Sciences, 1977, 66, 1-19, which is incorporated herein by reference. Pharmaceutically acceptable salts of the compounds disclosed herein include those derived from suitable inorganic and organic acids. Examples of pharmaceutically acceptable non-toxic acid addition salts are amino salts which are formed with mineral acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid, or formed by other methods in the art such as ion exchange. Other pharmaceutically acceptable salts include adipates, alginates, ascorbates, aspartates, benzenesulfonates, benzoates, bisulfates, borates, butyrates, camphorate, camphorsulfonates, citrates, cyclopentane propionates, digluconates, dodecyl sulfates, esylates, formates, fumarates, glucoheptonates, glycerophosphates, gluconate, hemisulfate, heptanoate, hexanoate, hydroiodide, 2-hydroxy-ethanesulfonate, lactobionate, lactate, laurate, lauryl sulfate, malates, maleates, malonates, methanesulfonates, 2-naphthalenesulfonates, nicotinates, nitrates, oleates, oxalates, palmitates, pamoate, a salt of pectinic acid, persulfates, 3-phenylpropionates, phosphates, picrates, pivalates, propionates, stearates, succinates, sulfates, tartrates, thiocyanates, p-toluenesulfonates, undecanoates, valerates, and the like.

### Preparation

The pirenzepine disclosed herein may be administered in any effective conventional dosage unit form including immediate release, sustained release, and timed release formulations, and pirenzepine may be administered together with a pharmaceutically acceptable carrier well known in the art in the following manner: oral, parenteral, topical, nasal, eye, intraperitoneal, intramuscular, intravenous, etc.

For oral administration, the compounds may be formulated into solid or liquid preparations such as capsules, pills, tablets, lozenges, troches, collosols, powders, solutions, suspensions, or emulsions, and may be prepared according to methods known in the art for preparing pharmaceutical compositions. The solid unit dosage forms can be capsules, which can be of the ordinary hard or soft capsule form and contain, for example, surfactants, lubricants, and inert fillers such as lactose, sucrose, calcium phosphate, and corn starch.

In another embodiment, pirenzepine can be compressed into a tablet together with a conventional tablet base (e.g., lactose, sucrose, and corn starch) in combination with binders (e.g., acacia gum, corn starch, or gelatin), disintegrants (e.g. potato starch, alginic acid, corn starch and guar gum, gum tragacanth, and gum acacia) used to assist in disintegration and dissolution of tablet after administration, lubricants (e.g., talc, stearic acid or magnesium stearate, calcium stearate or zinc stearate) used to increase the flowability of the tablet granulate and prevent the tablet material from adhering to the surface of the tablet molds and punches, dyes, colorants, and flavoring agents (e.g., peppermint oil, wintergreen oil, or cherry flavor) that can improve the sensory properties of the tablets and make them more acceptable to patients. Suitable excipients for oral liquid dosage forms include dicalcium phosphate and diluents (such as water and alcohols (e.g., ethanol, benzyl alcohol and polyethylene glycol)), with or without the addition of pharmaceutically acceptable surfactants, suspending agents or emulsifiers. Various other substances may be present as coatings or otherwise used to change the physical form of the dosage unit. For example, tablets, pills or capsules may be coated with shellac, sugar or both.

Dispersible powders and granules are suitable for the preparation of aqueous suspensions. They provide the active ingredient in admixture with a dispersing or wetting agent, a suspending agent and one or more preservatives. Examples of suitable dispersing or wetting agents and suspending agents are those mentioned above. Additional excipients may also be present, such as those sweeteners, flavorings, and colorants described above.

Pirenzepine may also be in the form of an oil-in-water emulsion. The oil phase can be a vegetable oil, such as liquid paraffin, or a mixture of vegetable oils. Suitable emulsifiers may be (1) natural gums, such as gum acacia and gum tragacanth, (2) natural phospholipids, such as soybean phospholipid and lecithin, (3) esters or partial esters derived from fatty acids and hexitol anhydrides, for example, sorbitan monooleate, (4) a condensation product of the partial ester with ethylene oxide, such as polyoxyethylene sorbitan monooleate. The emulsion may also include sweeteners and flavoring agents.

An oily suspension may be formulated by suspending pirenzepine in a vegetable oil (e.g., peanut oil, olive oil, sesame oil, or coconut oil) or in a mineral oil (e.g., liquid paraffin). The oily suspension may contain a thickening agent such as beeswax, hard paraffin or cetyl alcohol. The suspension may also contain one or more preservatives such as ethyl or n-propyl p-hydroxybenzoate; one or more colorants; one or more flavoring agents; and one or more sweeteners, such as sucrose or saccharin.

Syrups and elixirs may be formulated with sweeteners such as glycerin, propylene glycol, sorbitol, or sucrose. Such formulations may also include demulcents and preservatives (such as methyl paraben and propyl paraben) as well as flavoring and coloring agents.

The pirenzepine may also be administered parenterally at an injectable dose of the compound, i.e., subcutaneously, intravenously, intraocularly, intrasynovially, intramuscularly or intraperitoneally. The injectable dose is preferably in a physiologically acceptable diluent containing a pharmaceutical carrier. The pharmaceutical carrier can be a sterile liquid or a mixture of liquids. The liquid is, for example, water, saline, aqueous glucose and related sugar solutions, alcohols (e.g., ethanol, isopropanol or cetyl alcohol), diols (such as propylene glycol or polyethylene glycol), glycerol ketals (such as 2,2-dimethyl-1,1-dioxolane-4-methanol), ethers such as polyethylene glycol (400), oils, fatty acids, fatty acid esters or fatty acid glycerides or acetylated fatty acid glycerides, with or without the addition of pharmaceutically acceptable surfactants (such as soaps or detergents), suspending agents (such as pectin, carbomer, methylcellulose, hypromellose or carboxymethylcellulose), or emulsifiers and other pharmaceutical auxiliaries.

Exemplary oils that may be used in the parenteral formulations disclosed herein are those derived from petroleum, animal, vegetable or synthetic sources, such as peanut oil, soybean oil, sesame oil, cottonseed oil, corn oil, olive oil, petrolatum, and mineral oil. Suitable fatty acids include oleic acid, stearic acid, isostearic acid and myristic acid. Suitable fatty acid esters are, for example, ethyl oleate and isopropyl myristate. Suitable soaps include alkali metal, ammonium, and triethanolamine salts of fatty acids, and suitable detergents include cationic detergents such as dimethyl dialkyl ammonium halides, alkyl pyridinium halides, and alkylamine acetate; anionic detergents such as alkyl sulfonates, aryl sulfonates and olefin sulfonates, alkyl sulfates and alkyl sulfosuccinates, olefin sulfates and olefin sulfosuccinates, ether sulfates and ether sulfosuccinates, as well as monoglyceride sulfates and monoglycerides sulfosuccinates; nonionic detergents, such as fatty amine oxides, fatty acid alkanolamides, and poly(oxyethylene-oxypropylene), or ethylene oxide copolymers or propylene oxide copolymers; and amphoteric detergents such as alkyl-beta-aminopropionic acid salts and 2-alkylimidazolinium quaternary ammonium salts, and mixtures thereof.

Exemplary surfactants for parenteral formulations are polyethylene sorbitan fatty acid esters (e.g., sorbitan monooleate), and a high molecular weight adduct of ethylene oxide with a hydrophobic matrix (formed by condensation of propylene oxide and propylene glycol).

Pirenzepine may be in the form of a sterile aqueous suspension for injection. Such suspensions can be formulated according to known methods using suitable dispersing or wetting agents and suspending agents such as sodium carboxymethylcellulose, methylcellulose, hypromellose, sodium alginate, polyvinylpyrrolidone, gum tragacanth and gum acacia; dispersing or wetting agents, which may be natural phospholipids (e.g., lecithin), condensation products of alkylene oxides with fatty acids (e.g., polyoxyethylene stearate), condensation products of ethylene oxide with long-chain fatty alcohols (e.g. polyoxyethylene (17) cetyl alcohol), condensation products of ethylene oxide with partial esters derived from fatty acids and hexitols (e.g. polyoxyethylene sorbitol monooleate), or condensation products of ethylene oxide with partial esters derived from fatty acids and hexitol anhydrides (e.g., polyoxyethylene sorbitan monooleate).

The sterile injectable formulation may also be a sterile solution or suspension for injection in a nontoxic parenterally acceptable diluent or solvent. Useful diluents and solvents are, for example, water, Ringer's solution, isotonic sodium chloride solution and isotonic glucose solution. In addition, sterile, fixed oils are conventionally employed as a solvent or suspending medium. In this regard, any bland fixed oil may be employed including synthetic mono- or diglycerides. In addition, fatty acids such as oleic acid can be used in the preparation of injections.

For the above dosage forms, the amount of pirenzepine or a pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000 mg, based on pirenzepine.

### Treatment Kit

In other embodiments, also disclosed herein is a kit for conveniently and effectively implementing the method or use according to the present disclosure. Typically, the pack or kit comprises one or more containers filled with pirenzepine or a pharmaceutically acceptable salt thereof disclosed herein. This kit is particularly suitable for delivery in solid oral forms such as tablets or capsules. Such a kit preferably includes a number of unit doses, and may also include cards having a dose scheduled in their intended application order. If desired, a memory aid may be provided, for example, in the form of a number, letter or other indicia, or via a calendar insert indicating the number of days in the treatment schedule to which the dose can be given. Optionally associated with such container(s) may be a notice in the form prescribed by a governmental agency regulating the manufacture, use or sale of a pharmaceutical product, which notice reflects the approval of the production, application, or sale organization that is being used for human administration.

### DESCRIPTION OF THE DRAWINGS

In order to explain more clearly the examples disclosed herein or the technical solutions in the prior art, the drawings to be used in the description of the examples or the prior art will be briefly described below. Obviously, the drawings in the following description are only some examples of the invention. For those skilled in the art, other drawings may also be obtained based on these drawings without paying any creative labor.

FIG. 1 is a graph showing the survival curves of mice in each group with sepsis shock induced by low concentration of endotoxin (75 mg/kg) provided in the examples disclosed herein;

FIG. 2 is a graph showing the survival curves of mice in each group with sepsis shock induced by high concentration of endotoxin (150 mg/kg) provided in the examples disclosed herein;

FIG. 3 is a pathological diagram of a lung tissue of a normal control group provided in the examples disclosed herein;

FIG. 4 is a pathological diagram of a lung tissue of an endotoxin group provided in the examples disclosed herein;

FIG. 5 is a pathological diagram of a lung tissue of an atropine group provided by in the examples disclosed herein;

FIG. 6 is a pathological diagram of a lung tissue of a M1 receptor blocker pirenzepine group provided in the examples disclosed herein;

FIG. 7 is a pathological diagram of a lung tissue of an M2 receptor blocker AF-DX116 group provided in the examples disclosed herein;

FIG. 8 is a pathological diagram of a liver tissue of a normal control group provided in the examples disclosed herein;

FIG. 9 is a pathological diagram of a liver tissue of an endotoxin group provided in the examples disclosed herein;

FIG. 10 is a pathological diagram of a liver tissue of an atropine group provided in the examples disclosed herein;

FIG. 11 is a pathological diagram of a liver tissue of a M1 receptor blocker pirenzepine group provided in the examples disclosed herein;

FIG. 12 is a pathological diagram of a lung tissue of a M2 receptor blocker AF-DX116 group provided in the examples disclosed herein;

FIG. 13 is a survival curve of mice showing the effect of different concentrations of pirenzepine on mice with sepsis.

### EMBODIMENTS

To make the objectives, technical solutions, and advantages of the embodiments disclosed herein clearer, the following clearly and fully describes the technical solutions in the examples disclosed herein with reference to the accompanying drawings in the examples disclosed herein. Obviously, the described examples are part of the embodiments disclosed herein, not all of the embodiments. The elements and features described in one drawing or one embodiment disclosed herein may be combined with elements and features shown in one or more other drawings or embodiments. It should be noted that for the sake of clarity, illustrations and descriptions of components and processes known to those of ordinary skill in the art that are not relevant to the present disclosure are omitted from the drawings and description. All other embodiments obtained by persons of ordinary skill in the art based on the examples disclosed herein without creative efforts shall fall within the protection scope of the invention.

Chinese name disclosed herein:

[symbol]

Chinese alias: [symbol] ; [symbol] ; [symbol] ; [symbol] ;

[symbol]

English alias:

### Pirenzepine;

11-[(4-methylpiperazin-1-yl)acetyl]-5,11-dihydro-6H-pyrido[2,3-b][1,4]benzodiazepin-6-one; or 1-methyl-4-[2-oxo-2-(6-oxo-5,6-dihydro-11H-pyrido[2,3-b][1,4]benzodiazepin-11-yl)ethyl]piper azinediium.

Molecular Formula: C₁₉H₂₃N₅O₂

Molecular weight: 353.4171

Melting point: 243° C. (decomposition)

Boiling point: 541.7° C. at 760 mmHg

Flash point: 281.4° C.

Vapor pressure: 8.44E-12 mmHg at 25° C.

Properties: white crystalline powder, odorless, bitter taste.

Solubility: soluble in water, formic acid, insoluble in methanol, very easily soluble in anhydrous ethanol.

At present, the clinical indication of the drug is gastric ulcer. The usage and dosage in the treatment of sepsis diseases according to the present disclosure are as follows: the dosage of pirenzepine is 18-150 mg per day, oral administration for 1-3 times per day, and it can also be given once or several times for muscle/intravenous injection, and the course of treatment depends on the statute of the condition.

The following test examples illustrate the use of pirenzepine in the treatment of sepsis diseases.

### Test Example

Objective:

To explore whether non-selective M cholinergic receptor blocker atropine, selective M1 cholinergic receptor blocker pirenzepine, and selective M2 cholinergic receptor blocker AF-DX116 (English alias: otenzepad; 11-[[2-[(Diethylamino)methyl]-1-piperidinyl]acetyl]-5,11-dihydro-6H-pyrido[2,3-b][1,4]benzodi azepin-6-one) have a therapeutic effect on endotoxin-induced sepsis, and explore the relevant mechanism of action from changes in concentration of TNF-α in serum and pathological changes in liver and lung tissues, so as to screen out drugs that can be widely used for the treatment of sepsis clinically.

Methods:

The model of septic shock in mice was induced by intraperitoneal injection of a toxic dose of 75 mg/kg endotoxin. The effects of atropine, M1 receptor blocker pirenzepine, and M2 receptor blocker AF-DX116 on the survival rate of mice with sepsis shock were observed in groups (50 mice were randomly divided into 5 groups with 10 animals in each group). The model of sepsis shock in mice was induced by intraperitoneal injection of a total lethal dose of 150 mg/kg endotoxin. The effects of atropine, pirenzepine, and AF-DX116 on survival time of mice with sepsis shock were observed. The model of sepsis shock in rats was induced by 75 mg/kg endotoxin; blood was taken at 0, 30, 90, and 150 minutes after establishment of the sepsis shock model to observe the effects of atropine, pirenzepine and AF-DX116 on concentration of TNF-α in serum of rats, and liver and lung tissues were taken to observe the effects of atropine, pirenzepine and AF-DX116 on pathological changes of the liver and lungs of rats with sepsis shock under a microscope. Mice injected intraperitoneally with placebo in place of endotoxin were used as a normal control group.

Results:

Survival rate was observed in mice with sepsis shock induced by 75 mg/kg endotoxin: the survival rate of mice in the normal control group was 100%; the survival rate of mice in the endotoxin group was 40%; the survival rate of mice after the atropine treatment was 70%; the survival rate of mice after treatment with the M1 blocker pirenzepine was 80%; the survival rate of mice after treatment with the M2 blocker AF-DX116 was 10%. In the mice with septic shock induced by 150 mg/kg endotoxin, except that the mice in the normal control group did not die, all mice in the other groups died. The survival time of each group was observed: the average survival time of mice after pirenzepine treatment (44.6±11.96 hours) was significantly longer than that in the endotoxin group (29.15±6.84 hours) (p<0.05); the average survival time of mice after AF-DX116 treatment (16.02±2.06 hours) was significantly shorter than that in the endotoxin group (29.15±6.84 hours) (p<0.05); the average survival time of mice after atropine treatment (37.15±9.27 hours) was significantly longer than that in the endotoxin group (29.15±6.84 hours) (p<0.05).

After atropine treatment, serum TNF-α concentrations in rats were significantly lower than those in the endotoxin-untreated group at 0, 30, 90, and 150 minutes (p<0.05). After M1 receptor blocker pirenzepine treatment, serum TNF-α concentrations in rats were significantly lower than those in the endotoxin-untreated group at 0, 30, 90, and 150 minutes (p<0.05). Observed under microscope, the lung tissue of rats with septic shock showed obvious destruction of lung parenchyma, telangiectasias, and pulmonary interstitial infiltration of polymorphonuclear cells. The congestion, exudation, and necrosis were significantly reduced after treatment with atropine and pirenzepine. After the application of AF-DX116, the destruction of the tissue was aggravated, and obvious interstitial hyperemia, broadening, and exudation of a large number of inflammatory cells were observed. After endotoxin administration, hepatocytes were arranged disorderly, hepatocytes were flaky necrosis, hepatic sinusoids were widened, and hyperemia occurred. After atropine treatment and pirenzepine treatment, the lesions were all relieved, hepatic cells were arranged more neatly, and cell edema and necrosis were alleviated. In the AF-DX116 group, the arrangement of hepatocytes was more disordered than the endotoxin group, and cell necrosis was more serious.

Conclusion:

Atropine and pirenzepine significantly increase the survival rate of rats with endotoxin-induced sepsis shock, prolong survival time, reduce concentration of serum inflammatory factor TNF-α, and reduce inflammation and necrosis of liver and lung tissues.

### Determination of Effect on Survival Rate and Time of Survival Mice with Sepsis

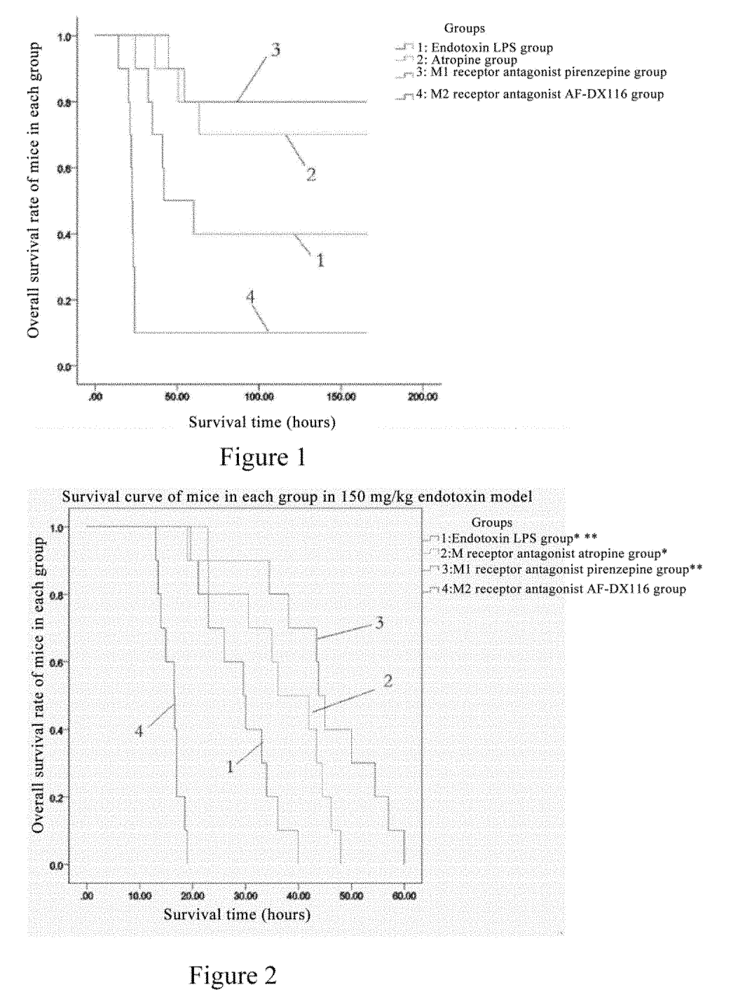

The model of sepsis shock in mice was induced with 75 mg/kg endotoxin. The survival of the mice was shown in FIG. 1. The normal control group: all survived; the endotoxin group: 4 survived and 6 died; the M1 blocker pirenzepine group: 8 survived and 2 died; the M2 blocker AF-DX116 group: 1 survived and 9 died; the atropine group: 7 survived and 3 died. Based on the above results, the survival curves were plotted in FIG. 1: 3 represents the M1 receptor blocker pirenzepine group; 2 represents the atropine group; 1 represents the endotoxin group; and 4 represents the M2 receptor blocker AF-DX116 group. The M1 receptor blocker pirenzepine group significantly reduced the mortality of mice with sepsis.

The septic shock in mice was induced with 150 mg/kg endotoxin and the survival curves were plotted according to the survival time of mice in each group. See FIG. 2: 1 represents the endotoxin group; 3 represents the M1 receptor blocker pirenzepine group; 4 represents the M2 receptor blocker AF-DX116 group; and 2 represents the atropine group. M1 receptor blocker pirenzepine significantly reduced the mortality of mice with sepsis.

### Determination of Serum TNF-α Concentration

Concentration of TNF-α in serum of rats was detected at 0 min after sepsis shock was reached in rats. Concentration of TNF-α in serum of rats in a normal control group was (159.36±50.40) pg/mL; concentration of TNF-α in serum of rats in an endotoxin group was (641.05±24.75) pg/mL; the concentration of TNF-α in serum of rats in the endotoxin group was significantly higher than that in the normal control group (159.36±50.40) pg/mL, and the difference was statistically significant (p<0.05); the concentration of TNF-α in serum of rats in the group of the selective M1 cholinergic receptor antagonist pirenzepine was (378.12±17.87) pg/mL, the concentration of TNF-α in serum of rats in the pirenzepine group was significantly lower than that in the endotoxin group (641.05±24.75) pg/mL, and the difference was statistically significant (p<0.05); the concentration of TNF-α in serum of rats in the group of the selective M2 cholinergic receptor antagonist AF-DX116 was (706.19±6.00) pg/mL, the concentration of TNF-α in serum of rats in the AF-DX116 group was significantly higher than that in the endotoxin group (641.05±24.75) pg/mL, and the difference was statistically significant (p<0.05); the concentration of TNF-α in serum of rats in the atropine group was (347.87±39.24) pg/mL, concentration of TNF-α in serum of rats in the atropine group was significantly lower than that in endotoxin group (641.05±24.75) pg/mL, and the difference was statistically significant (p<0.05); the concentration of TNF-α in serum in the atropine group was lower than that of the pirenzepine group (378.12±17.87) pg/mL, and the difference was not statistically significant (p>0.05).

Concentration of TNF-α in serum of rats was detected at 30 minutes after sepsis shock was reached in rats. Concentration of TNF-α in serum of rats in the normal control group was (223.41±27.42) pg/mL; concentration of TNF-α in serum of rats in the endotoxin group was (677.95±47.05) pg/mL, the concentration of TNF-α in serum of rats in the endotoxin group was significantly higher than that in the normal control group (223.41±27.42) pg/mL, and the difference was statistically significant (p<0.05); concentration of TNF-α in serum of rats in the group of the selective M1 cholinergic receptor antagonist pirenzepine was (418.19±22.75) pg/mL, the concentration of TNF-α in serum of rats in the pirenzepine group was significantly lower than that in the endotoxin group (677.95±47.05) pg/mL, and the difference was statistically significant (p<0.05); concentration of TNF-α in serum of rats in the group of the selective M2 cholinergic receptor antagonist AF-DX116 was (757.48±51.40) pg/mL, the concentration of TNF-α in serum of rats in the AF-DX116 group was significantly higher than that in the endotoxin group (677.95±47.05) pg/mL, and the difference was statistically significant (p<0.05); concentration of TNF-α in serum of rats in the atropine group was (421.51±13.37) pg/mL, the concentration of TNF-α in serum of rats in the atropine group was significantly lower than that in the endotoxin group (677.95±47.05) pg/mL, and the difference was statistically significant (p<0.05); the concentration of TNF-α in serum of rats in the atropine group was higher than that in the pirenzepine group (418.19±22.75) pg/mL, and the difference was not statistically significant (p>0.05).

Concentration of TNF-α in serum of rats in each group was detected at 90 minutes after septic shock was reached in rats. The concentration of TNF-α in serum of rats in the normal control group of atropine was (166.86±28.80) pg/mL; the concentration of TNF-α in serum of rats in the endotoxin group was (716.11±24.41) pg/mL, the concentration of TNF-α in serum of rats in the endotoxin group was significantly higher than that in the normal control group (166.86±28.80) pg/mL, and the difference was statistically significant (p<0.05); the concentration of TNF-α in serum of rats in the group of the selective M1 cholinergic receptor antagonist pirenzepine was (446.35±33.04) pg/mL, the concentration of TNF-α in serum of rats in the pirenzepine group was lower than that in the endotoxin group (716.11±24.41) pg/mL, and the difference was statistically significant (p<0.05); the concentration of TNF-α in serum of rats in the group of the selective M2 cholinergic receptor antagonist AF-DX116 was (857.21±74.14) pg/mL, the concentration of TNF-α in serum of rats in the AF-DX116 group was significantly higher than that in the endotoxin group (716.11±24.41) pg/mL, and the difference was statistically significant (p<0.05); the concentration of TNF-α in serum of rats in the atropine group was (598.54±48.85) pg/mL, the concentration of TNF-α in serum of rats in the atropine group was significantly lower than that in the endotoxin group (716.11±24.41) pg/mL, and the difference was statistically significant (p<0.05); the concentration of TNF-α in serum of rats in the atropine group was higher than that in the pirenzepine group (446.35±33.04) pg/mL, and the difference was statistically significant (p<0.05).

Concentration of TNF-α in serum of rats was detected at 150 minutes after sepsis shock was reached in rats. The concentration of TNF-α in serum of rats in the normal control group was (161.47±9.75) pg/mL; the concentration of TNF-α in serum of rats in the endotoxin group was (737.00±16.38) pg/mL, the concentration of TNF-α in serum of rats in the endotoxin group was significantly higher than that in the normal control group (161.47±9.75) pg/mL, and the difference was statistically significant (p<0.05); the concentration of TNF-α in serum of rats in the group of the selective M1 cholinergic receptor antagonist pirenzepine was (539.49±19.39) pg/mL, the concentration of TNF-α in serum of rats in the pirenzepine group was significantly lower than that in the endotoxin group (737.00±16.38), and the difference was statistically significant (p<0.05); the concentration of TNF-α in serum of rats in the group of the selective M2 cholinergic receptor antagonist AF-DX116 was (932.22±17.25) pg/mL, the concentration of TNF-α in serum of rats in the AF-DX116 group was significantly higher than that in the endotoxin group (737.00±16.38) pg/mL, and the difference was statistically significant (p<0.05); the concentration of TNF-α in serum of rats in the atropine group was (611.30±12.52) pg/mL, the concentration of TNF-α in serum of rats in the atropine group was significantly lower than that in the endotoxin group (737.00±16.38) pg/mL, and the difference was statistically significant (p<0.05); the concentration of TNF-α in serum of rats in the atropine group was higher than that in the pirenzepine group (539.49±19.39) pg/mL, and the difference was statistically significant (p<0.05).

TABLE 1
Table 1 is a comparison table of detected concentrations of TNF-α in serum.

| | 0 min | 30 min | 90 min | 150 min |
|---|---|---|---|---|
| the normal control | 159.36 ± 50.40 | 223.41 ± 27.42 | 166.86 ± 28.80 | 161.47 ± 9.75 |
| group | | | | |
| the endotoxin group | 641.05 ± 24.75 | 677.95 ± 47.05 | 716.11 ± 24.41 | 737.00 ± 16.38 |
| the M1 blocker | 378.12 ± 17.87^{a} | 418.19 ± 22.75^{c} | 446.35 ± 33.04^{e} | 539.49 ± 19.39^{g} |
| pirenzepine group | | | | |
| the M2 blocker | 706.19 ± 6.00 | 757.48 ± 51.40 | 857.21 ± 74.14 | 932.22 ± 17.25 |
| AF-DX116 group | | | | |
| the atropine group | 347.87 ± 39.24^{b} | 421.51 ± 13.37^{d} | 598.54 ± 48.85^{f} | 611.30 ± 12.52^{h} |

### Pathological Change in Liver and Lung Tissues Under Microscope

### 1. Pathological Change in a Lung Tissue Under Microscope

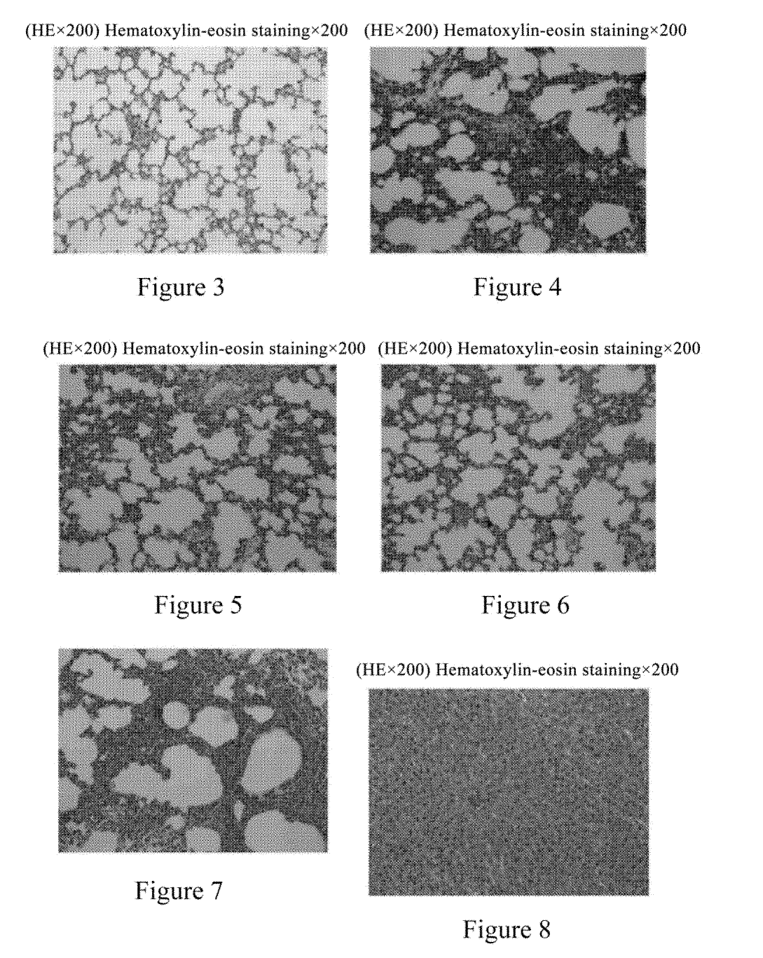

The normal control group (FIG. 3) showed an intact structure of the lung tissue, no edema and inflammation in the alveolar septum, and clear alveolar space; the endotoxin group (FIG. 4) showed pathological changes of the lung tissue under microscope: the sizes of the alveoli were different, some of the alveolar wall were ruptured with emphysema, some alveoli were collapsed, the alveolar wall was widened, alveolar capillaries were expanded, and pulmonary interstitial polymorphonuclear cell infiltration was observed; the atropine group (FIG. 5) showed pathological changes of the lung tissue under microscope: compared with the endotoxin group, the exudation was significantly reduced, and alveolar septum widening was reduced; the M1 receptor blocker pirenzepine group (FIG. 6) showed pathological changes of the lung tissue under microscope: compared with the endotoxin group, the exudation was significantly reduced, and edema and inflammation in the alveolar septum were reduced; compared with the atropine group, the inflammation was slightly reduced; the M2 receptor blocker AF-DX116 group (FIG. 7) showed pathological changes of the lung tissue under microscope: compared with the endotoxin group, pathological changes were aggravated, and the alveolar septum was significantly congested and widened, extensive inflammatory cells exuded, the alveolar space was narrow obviously, and the alveoli were collapsed.

### 2. Pathological Change in a Liver Tissue Under Microscope

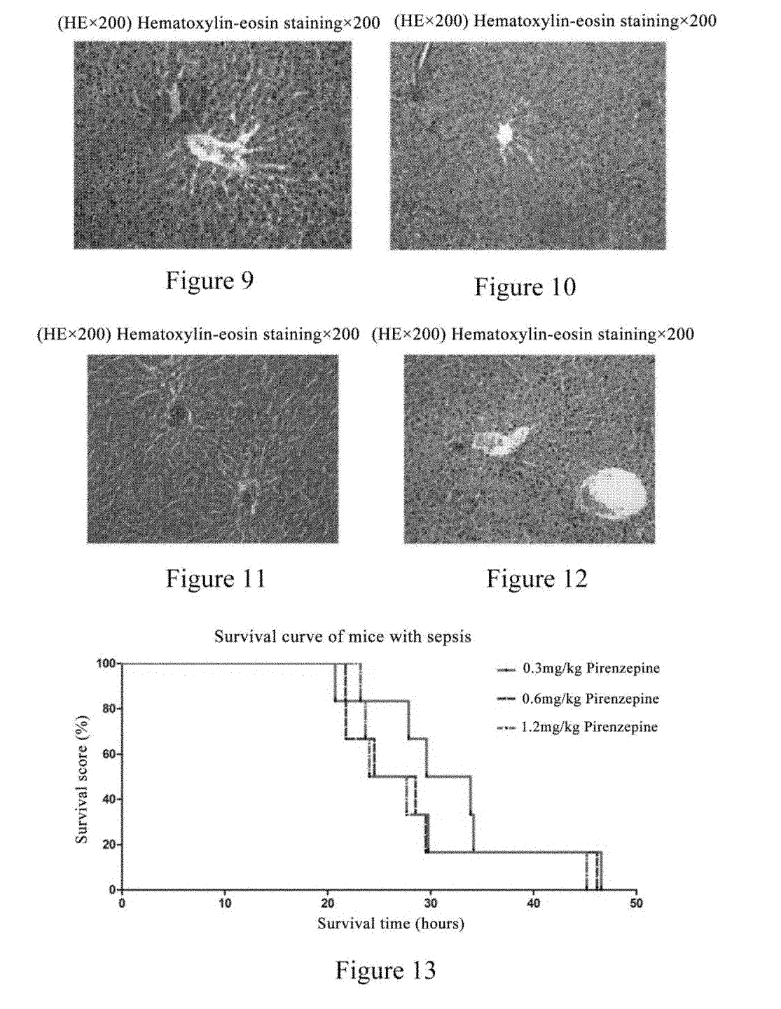

The normal control group (FIG. 8) showed the following liver morphology under microscope: the normal hepatic cell was arranged radially around the central vein, the hepatic tissue was dense, the outline of hepatic lobule was clear, and the hepatic cord was arranged neatly; the endotoxin group (FIG. 9) showed pathological changes of the liver tissue under microscope: the hepatic cell was arranged disorderly and swollen, and flaky necrosis was observed; the hepatic sinus was widened and congested, and polymorphonuclear cell infiltration was observed; the atropine group (FIG. 10) showed pathological changes of the liver tissue under microscope: compared with the endotoxin group, the cells were arranged more neatly, the cells were arranged into cords, and the edema and necrosis of hepatocytes were all reduced; the M1 cholinergic receptor blocker pirenzepine group (FIG. 11) showed pathological changes of the liver tissue under microscope: compared with the endotoxin group, the lesions were reduced, the cells were arranged neatly and orderly, the hepatocytes were arranged radially around the blood vessels, and the hepatic sinus was slightly widened; the M2 cholinergic receptor blocker AF-DX116 group (FIG. 12) showed pathological changes of the liver tissue under microscope: the arrangement of hepatocytes was more disordered than that of the LPS group, and cell necrosis was more severe.

### Study on the Effects of Different Doses of Pirenzepine

C57BL/6 mice were pretreated with different concentrations of M1 receptor blocker pirenzepine (0.3 mg/kg, 0.6 mg/kg, 1.2 mg/kg) by intraperitoneal injection. After 30 minutes, LPS (75 mg/kg) was injected intraperitoneally to produce a mouse sepsis model. We found that a low-dose pretreatment with pirenzepine can prolong the survival time of mice with sepsis, with low-dose pirenzepine treatment being superior, but there was no statistically significant difference between them, wherein the mean survival time in 0.3 mg/kg pirenzepine group was 29.600±3.686 hours, the survival time in 0.6 mg/kg pirenzepine group was 24.530±4.140 hours, and the average survival time in 1.2 mg/kg pirenzepine group was 24.030±2.437 hours, and there was no statistically significant difference between groups, see FIG. 13.

In the foregoing examples disclosed herein, the sequence numbers of the examples are only for convenience of description, and do not represent the advantages and disadvantages of the examples. The description of each example has its own emphasis. For the parts that are not described in detail in one example, reference may be made to the description of other examples.

In the embodiments of the apparatus and method of the invention, it is apparent that each component or step can be decomposed, combined, and/or recombined after it is decomposed. These decompositions and/or recombinations should be considered equivalent solutions of the invention. Meanwhile, in the foregoing description of the specific examples disclosed herein, the features described and/or illustrated for one example can be used in one or more other examples in the same or similar manners, combined with features in other examples, or substitute for features in other examples.

It should be emphasized that the term “comprising” is used herein to refer to the presence of a feature, element, step or component, but does not preclude the presence or addition of one or more other features, elements, steps or components.

Finally, it should be stated that while the disclosure and its advantages have been described in detail above, it should be understood that various changes, substitutions and alterations can be made without departing from the spirit and scope disclosed herein as defined by the appended claims. Moreover, the scope disclosed herein is not limited to the specific examples of the processes, apparatuses, means, methods, and steps described in the specification. Those of ordinary skill in the art will readily understand from the disclosure disclosed herein that existing and future processes, apparatuses, means, methods, or steps for performing substantially the same functions as or obtaining substantially the same results as the corresponding examples described herein may be used in accordance with the present invention. Accordingly, the appended claims are intended to include within their scope such processes, apparatuses, means, methods, or steps.

1-10. (canceled)

11. A method of treating sepsis, comprising administering pirenzepine or a pharmaceutically acceptable salt thereof.

12. The method of claim 11, wherein pirenzepine or the pharmaceutically acceptable salt thereof is administered by intraperitoneal injection, intramuscular injection, intravenous injection or oral administration.

13. The method of claim 12, wherein pirenzepine or the pharmaceutically acceptable salt thereof is administered by intraperitoneal injection in an amount per injection of 0.1-20 mg/kg, based on pirenzepine.

14. The method of claim 12, wherein pirenzepine or the pharmaceutically acceptable salt thereof is administered by intraperitoneal injection in an amount per injection of 0.3 mg/kg, 0.6 mg/Kg, 1.2 mg/kg, 1.5 mg/kg, 2 mg/kg, 3 mg/kg, 5 mg/kg, 7 mg/kg, 9 mg/kg, 11 mg/kg, 13 mg/kg, 15 mg/kg, 17 mg/kg, or 20 mg/kg, based on pirenzepine.

15. The method of claim 12, wherein pirenzepine or the pharmaceutically acceptable salt thereof is administered by intramuscular or intravenous injection in an amount of 10-1000 mg/day, once daily or divided into several doses, based on pirenzepine.

16. The method of claim 12, wherein pirenzepine or the pharmaceutically acceptable salt thereof is administered by intramuscular or intravenous injection in an amount of 18-150 mg/day, once daily or divided into several doses, based on pirenzepine.

17. The method of claim 12, wherein pirenzepine or the pharmaceutically acceptable salt thereof is orally administered in an amount of 10-1000 mg/day, 1-3 times per day, based on pirenzepine.

18. The method of claim 12, wherein pirenzepine or the pharmaceutically acceptable salt thereof is orally administered in an amount of 18-150 mg/day, 1-3 times per day, based on pirenzepine.

19. The method of claim 12, wherein pirenzepine or the pharmaceutically acceptable salt thereof is in a unit dosage form of a solid or liquid formulation.

20. The method of claim 19, wherein the solid or liquid formulation is a capsule, a pill, a tablet, a lozenge, a troche, a collosol, a powder, a solution, a suspension or an emulsion.

21. The method of claim 19, wherein the amount of pirenzepine or the pharmaceutically acceptable salt thereof in the unit dosage form is 0.1-1000 mg, based on pirenzepine.

22. The method of claim 19, wherein the amount of pirenzepine or the pharmaceutically acceptable salt thereof in the unit dosage form is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 mg, based on pirenzepine.

23. The method of claim 20, wherein the amount of pirenzepine or the pharmaceutically acceptable salt thereof in the unit dosage form is 0.1-1000 mg, based on pirenzepine.

24. The method of claim 20, wherein the amount of pirenzepine or the pharmaceutically acceptable salt thereof in the unit dosage form is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 mg, based on pirenzepine.
